(19) United States
(12) Patent Application Publication
(10) Pub. No.: US 2007/0021739 A1
(43) Pub. Date: Jan. 25, 2007

(54) Inter-atrial Transseptal Laser Puncture (TLP) Procedure

(75) Inventor: Helmut Weber, Munich (DE)

Correspondence Address: HELMUT WEBER, MD, ZENNERSTR. 16, MUNICH, D-81379, DE

(73) Assignee: LASCOR GMBH, Munich (DE)

(21) Appl. No.: 11/161,124

(22) Filed: Jul. 24, 2005

(51) Int. Cl. A61B 18/18

(52) U.S. Cl. 606/15

(57) ABSTRACT

A method for puncture of the inter-atrial septum of a heart is performed by means of a laser catheter assembly comprising at least one longitudinal sheath and an optical fiber connectable to a laser energy source. The components of the laser catheter assembly are perveneously introduced and advanced into the right atrium until the sheath is in intimate contact with the septal wall and laser light is applied via said optical fiber and emitted on the tissue located in front of the distal end of the fiber so as to create a small hole in said inter-atrial septum.

Definition List 1

| Term | Definition |
|---|---|
| FO | fossa ovalis |
| IAS | inter-atrial septum |
| RA | right atrium |
| RV | right ventricle |
| LA | left atrium |

This invention relates to a method for puncture of the inter-atrial septum of a heart, particularly of a human heart, and a laser catheter assembly comprising at least one longitudinal sheath and an optical fiber housed inside the sheath.

The interior of a human heart consists of four distinct chambers. The upper chambers are known as right and left atrium and the lower chambers as right and left ventricles. The septum, a fixed central muscular wall, separates the right and left cavities from each other. By contraction of a muscle called myocardium, the heart pumps the deoxygenated blood to the pulmonary arteries and the oxygenated blood to the aorta. The contraction of the heart is performed with a certain heart rate and controlled by electrical impulses which stimulate the heart. When the electrical stimulation of the heart muscle is affected, the heart rate shows arrhythmias which impair the quality of life of millions of people and restrict exercise capacity, is causing pain, breathlessness or fatigue and even death.

In order to cure these arrhythmias, catheter ablation of the endo-myocardium has been successfully applied in recent years, even when located in the left side of the heart [1]. Thereby, the laser catheter is introduced percutaneously through puncture of a vein in the groin and is advanced into the heart cavity and laser light is aimed at a defined area of the cardiac wall in order to coagulate the diseased tissue of the myocardium and to reduce or abolish electrical conduction in that pathological region of the cardiac wall. If diseased areas located in the left side of the heart are to be treated, the catheter can be inserted retrogradely, e.g. trans-aortal, or pervenously via the foramen ovale (also known as the fossa ovalis) which is a membrane located in the inter-atrial septum.

Transseptal catheterization is relatively uncomplicated if the fossa ovalis is open. However, a patent fossa ovalis can be identified only in 10 % to 25 % of people [2]. Therefore, needle puncture of an inter-atrial septum is the standard technique for transvenous introduction of catheters into the left side of the heart. When using this technique, the inter-atrial septum is routinely punctured with a long needle which is well known as Brockenbrough needle. A dilator is loaded with such a needle which is advanced to within 2 cm to 4 cm of the dilator tip. From the high right atrium position the dilator and needle are rotated towards the inter-atrial septum and slowly withdrawn under fluoroscopic guidance while assessing for a characteristic “jump” as the tip of the dilator is passed over the aortic knob and onto the fossa ovalis. Then the dilator is manipulated until the tip is in intimate contact with the fossa ovalis. Thereafter, the needle is pushed with a strike towards the left atrium whereby the inter-atrial septum is punctured. The advancement of the needle is then immediately halted. Under fluoroscopic control, a floppy guide wire is advanced through the needle in order to confirm successful puncture of the inter-atrial septum and access to the left atrium.

As compared to the retrograde aortical reproach, the transseptal technique can avoid inadvertent damages to the femoral artery, the coronary arteries or the aortic valve as well as thromboembolic events with cerebrovascular accidents. In addition, with the support by the almost motionless inter-atrial septum, it allows for a safer and easier manipulation of catheters in the left atrium providing a more stable position and endocardial contact of catheters. Thus, it contributes to the reduction of procedure duration, X-ray exposure times and increases success rate for catheter ablation of left sided arrhythmias [3].

However, complication rate of the routine transseptal procedure using a Brockenbrough needle is 2%-6%. These complications can be serious and life threatening, including atrial perforation, aortic perforation, pericardial tamponade and death mainly due to unintentional puncture of wrong structures [4-8]. For a better control of the procedure, transesophageal or intracardiac echocardiography is used as an adjunct to fluoroscopy to guide transseptal left heart catheterization [5, 6, 9]. However, in some cases transseptal needle puncture is not practical at all because of a resistant fibrous inter-atrial septum or for other anatomical reasons. Especially when a site-selective transseptal left atrial access as recently propagated is needed, needle puncture may be unsuitable and risky [10].

It is therefore an object of the present invention to provide a method for puncture of the inter-atrial septum which is less affecting and hurtful to the patient as well as to provide a catheter assembly for transseptal left heart catheterization which is easy to operate and less harmful to the patient.

The present invention provides a method and apparatus to performing a minimally invasive surgical (MIS) procedure by means of which a channel/hole in the inter-atrial septum may be produced temporarily, which is closing spontaneously after removal of the inserted catheter. It is a central principle of the present invention to provide a method for puncture of the inter-atrial septum of a heart by means of a laser catheter assembly comprising an optical fiber and a guide means for guiding the optical fiber to the region to be treated, wherein laser light is applied via the optical fiber and irradiated on the tissue located in front of the distal end of the fiber, thereby vaporizing the tissue and creating a small hole in the atrial septum. The catheter assembly preferably comprises at least one flexible sheath (like a dilator or another tube-like guide means) and an optical fiber which is preferably located inside the sheath. In comparison to the well known Brockenbrough needle technique, the transseptal laser puncture technique according to the invention has the major advantage that the flexibility of the optical fiber allows for easy advancement of the catheter assembly without stretching the dilator when passing the bifurcation region of the inferior vena cava, thereby avoiding discomfort or pain to the patient. Furthermore, there is only a minimal trauma caused by the small hole produced in the inter-atrial septum.

According to a preferred embodiment of the invention, the optical fiber is brought into intimate contact with the septal wall and advanced into the septal wall during laser application. Thereby, the septal wall can be punctured with a minimum trauma of the anatomic structure and a minimum discomfort of the patient.

Alternatively, the septal wall can also be penetrated merely mechanically by advancement of the fiber without application of laser light. The mechanical puncture technique is feasible only in case of a patent fossa ovalis or a very thin septal wall which is easy to penetrate.

The best location for puncture is the position of the fossa ovalis in the atrial septum because of its low wall thickness. Therefore, the catheter assembly is preferably located at the position of the fossa ovalis.

In case of laser application the optical fiber is preferably advanced (e.g. 3-5 mm) beyond a distal end of the sheath, in particular beyond a distal opening of a dilator.

The power of laser light applied to the septal tissue is preferably less than 20 W, preferably about 10 W. The application time is usually less than 5 s.

The optical fiber according to a preferred embodiment of the invention has a pointed tip or a plane distal light emitting surface which extends mainly along the longitudinal axis of the fiber. The fiber tip is preferably pointed.

Transseptal puncture can either be performed with a catheter assembly comprising only one sheath, e.g. a dilator and the optical fiber, or several sheaths nested inside or overriding each other respectively. Application of a single sheath is preferable in pediatric patients or when a less traumatic access is intended. The sheaths are preferably pre-shaped.

According to a special embodiment of the present invention, the catheter assembly comprises an inner sheath, an outer sheath overriding the inner sheath, a dilator which is located inside a lumen of the inner sheath and an optical fiber which is located inside a lumen of the dilator. The inner sheath may consist of a pre-shaped plastic tube with two consecutive curves of e.g. 45° following each other in a perpendicular plane. The outer sheath is overriding the inner sheath. It may consist of a plastic tube having e.g. a 45° curve at a distal portion thereof.

After penetration of the inter-atrial septum by means of the optical fiber, the one or several sheaths of the catheter means are preferably advanced over the fiber into the left atrium.

In case of incertitude as regards position of puncture, the optical fiber is preferably removed and the dilator is not advanced over the fiber into the left atrium, but kept in a stable position. While the dilator is sticking in the hole produced by the optical fiber in the septal wall, a blood sample can be obtained, and/or pressure measurement can be performed via the dilator, as well as contrast medium injected in order to check the site of penetration.

Eventually, the sheath can be loaded with a guide wire which is advanced beyond the end hole of the dilator (into the left atrium if placed correctly), whereby location of the puncture hole can be definitively confirmed under X-ray surveillance. Now, the sheath(s) can be advanced over the guide wire into the left atrium.

Numerous other advantages and features of the present invention will become readily apparent from the following detailed description of the invention and the embodimen ts thereof, from the claims and from the accompanying drawings.

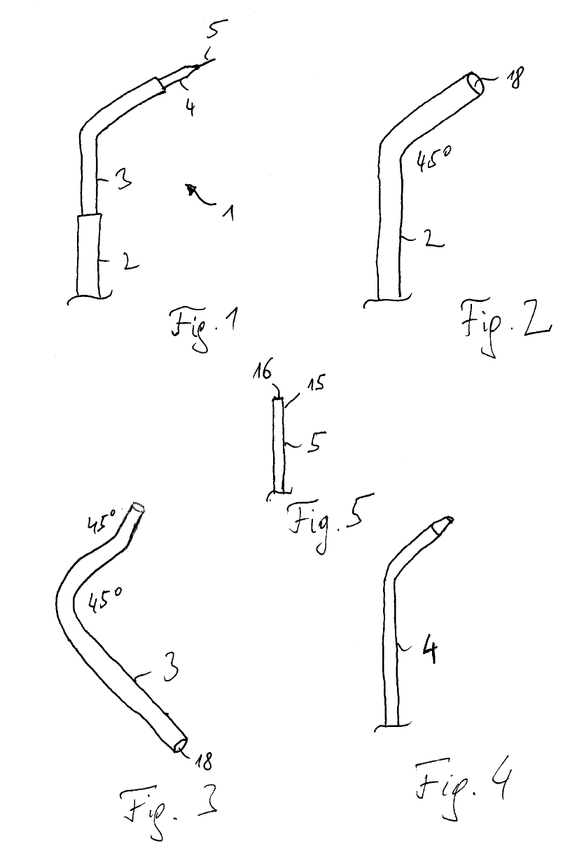

FIG. 1 is a schematic view of a catheter assembly having three sheaths according to a first embodiment of the invention.

FIG. 2 is a schematic view of a distal portion of an outer catheter sheath which is pre-shaped.

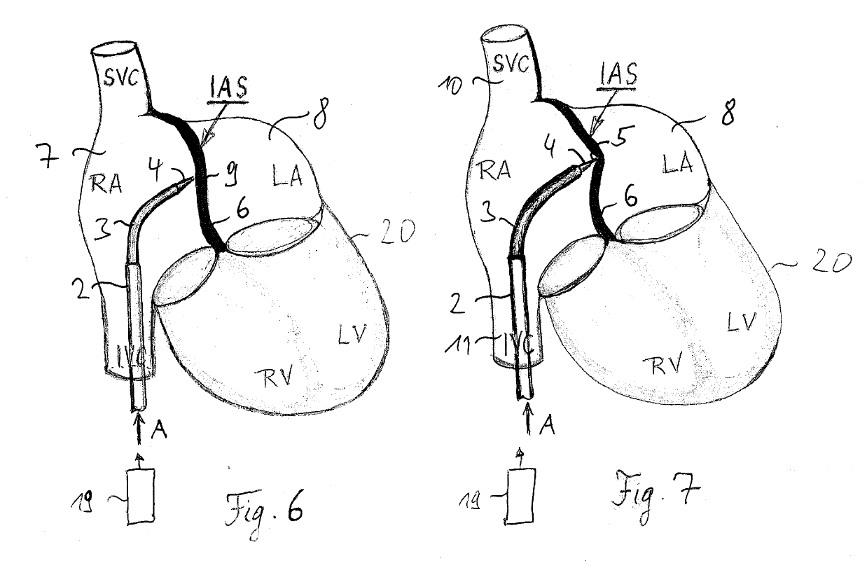

FIG. 3 is a schematic view of a distal portion of an inner catheter sheath which is pre-shaped.

FIG. 4 is a schematic view of a distal portion of a pre-shaped dilator.

FIG. 5 is a schematic view of a distal portion of an optical fiber.

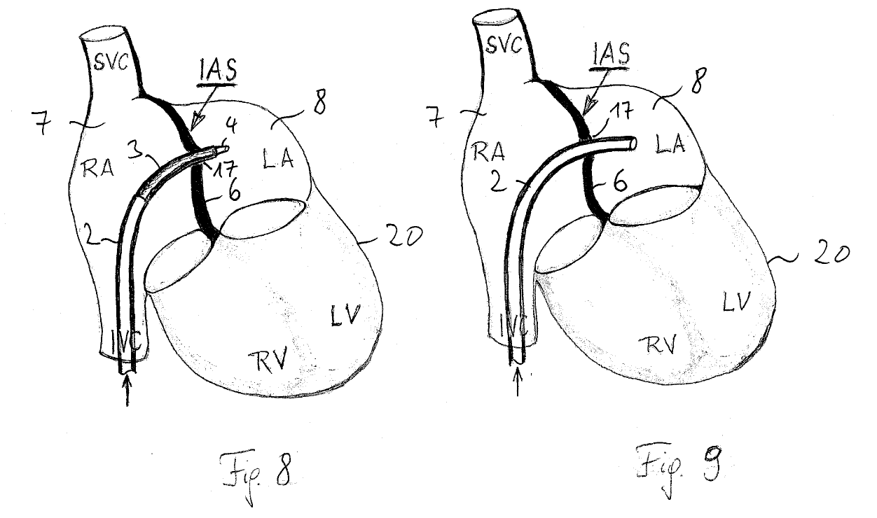

FIG. 6 is a schematic view of a human heart having the catheter assembly of FIG. 1 introduced therein.

FIG. 7 is a schematic view of a human heart with the laser catheter assembly of FIG. 1 being in intimate contact with the septal wall.

FIG. 8 is a schematic view of a human heart with a dilator and an inner sheath of the catheter assembly of FIG. 1 protruding into the left atrium.

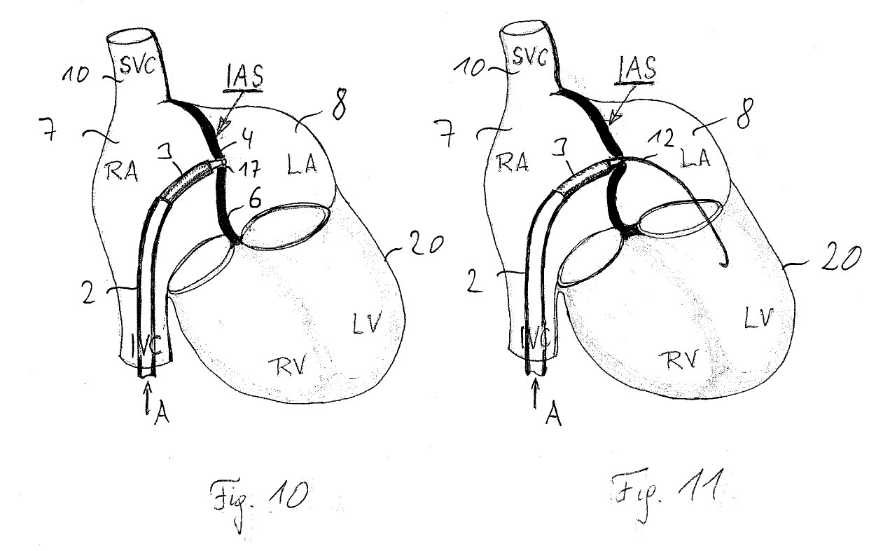

FIG. 9 is a schematic view of a human heart with an outer sheath of the catheter assembly of FIG. 1 protruding into the left atrium.

FIG. 10 is a schematic view of a human heart with the dilator tip of the catheter assembly of FIG. 1 sticking in the septal wall.

FIG. 11 is a schematic view of a human heart with a guide wire introduced into the left side of the heart.

FIG. 1 is a schematic view of a catheter assembly which is preferably used for puncture of the inter-atrial septum 6 (see FIG. 6) of a human heart 20. The catheter assembly 1 comprises an outer sheath 2 overriding an inner sheath 3 which is in turn overriding a dilator 4 having an inner lumen 18 (see FIG. 4) in which an optical fiber 5 is housed. The sheaths 2, 3, 4 and the optical fiber 5 are overriding each other and can be moved in longitudinal direction relatively to each other as well as twisted in a circumferential direction.

FIG. 2 is a schematic view of an outer sheath 2 which is pre-shaped at a distal portion thereof. Here, the outer sheath 2 consists of an 10 F (inner diameter) 50 cm long and 45° curved plastic tube. (For reasons of optimal manipulation characteristics an angle of about 45° is preferred, but other angles may be applied as well depending of the geometrical structures of the target location. The outer sheath 2 has an inner lumen which is denoted by reference numeral 18.

FIG. 3 shows an example of an inner sheath 3 which is pre-shaped at the distal portion thereof. The inner sheath 3 consists of an 8F, 60 cm long pre-shaped plastic tube with two consecutive curves of 45° located in two planes perpendicular to each other. The inner lumen of the sheath 3 is denoted by reference numeral 18.

FIG. 4 is a schematic view of a dilator 4 which is pre-shaped at a distal portion thereof. Here, the dilator 4 consists of a pre-shaped plastic tube having two curves of 45° each and a tapered tip. The dilator 4 has an inner lumen which is denoted by reference numeral 18. During operation, the dilator 4 is advanced several millimeters beyond the distal end hole of the guiding catheter 3 (inner sheath).

FIG. 5 is a schematic view of a distal portion 15 of an optical fiber 5 used for puncture of the septum. The optical fiber 5 has a light emitting surface 16 which is preferably pointed up to 80° or flat. Thereby the irradiated laser light is not, or only slightly diffused so that a very small hole can be produced in the septum 6.

FIGS. 6 to 8 are schematic views of a human heart showing different consecutive states of a laser catheter assembly of FIG. 1 during the transseptal puncture procedure. After venous puncture in the groin using the well-known Seldinger technique, a guide wire is advanced into the right atrium and the puncture needle is removed. Then, the previously saline-flushed (heparin 5000 IU/I) and assembled pre-shaped sheaths 2, 3 and the dilator 4 are introduced over the guide wire (not shown) and advanced via the inferior vena cava (IVC) into the high right atrium 7 (RA). Due to the resilience, “memory effect”, of the catheter material the preshaped inner sheath 3 orientates its tip towards the inter-atrial septum 6 (IAS). Under X-ray control (or other appropriate surveillance), the tip of the dilator 4 is brought in intimate contact with the septal wall 6, preferably in the area of the fossa ovalis 9. The guide wire (not shown) is replaced by an optical fiber 5.

FIG. 6 shows the catheter assembly 1 positioned with a distal, intra-cardiac end in the mid of the right atrial cavity and with the tip of the transseptal dilator 4 oriented towards the central area of the inter-atrial septum 6.

FIG. 7 is a consecutive state of the catheter assembly 1, wherein the tip of the optical fiber 5 is advanced approximately 3 mm beyond the end hole of the dilator 4 and is slightly tenting the inter-atrial septal wall 6 towards the left atrium 8 (LA). In this state, laser light is applied to the septal tissue by means of a laser energy source 18 which is operatively connected to the optical fiber 5.

During laser light application, the catheter assembly with the optical fiber 5 is advanced towards the left atrium 8 until the fiber 5 penetrates the septal wall 6. After the optical fiber has reached the left atrium 8, dilator 4 and inner sheath 3 are advanced over the optical fiber 5 through the hole 17 inside the septum 6 into the left atrium 8. Then, the optical fiber 5 is instantaneously withdrawn.

FIG. 8 shows a state, in which the pre-shaped sheath 3 and the dilator 4 are positioned approximately 1 cm inside the left atrial cavity 8. In this position, the transseptal dilator 4 is slightly protruding from the end hole of the inner sheath 3.

After advancement of the dilator 4 and the inner sheath 3, also the outer sheath 2 is advanced into the left atrium 8. Then the dilator 4 and the inner sheath 3 are withdrawn from the outer sheath 2 which is now placed with its distal end in the left atrial cavity.

FIG. 9 shows the outer sheath 2 advanced through the inter-atrial septum 6. The outer sheath 2 is now filled and continuously flushed with heparinized (5000 IU/I) saline via a side arm of a haemostatic valve attached at a lower Luer gate (not shown) which is located at a proximal end of the sheath 2. The transseptal sheath 2 is now ready for left heart catheterization via its lumen 18 with catheters of various purposes, e.g. for ablation, intra-cardiac echocardiography, angio-cardioscopy, biopsy etc..

In most cases, a position for puncture in the region of the fossa ovalis may be found and confirmed under X-ray observation (or other appropriate surveillance). In case of incertitude as regards the position of puncture and because of the possible serious risks related with puncture of wrong structures, it is advisable to reconfirm if the tip of the dilator 4 is located at the proper position of the septal wall 6. A method for confirmation of the exact position of the dilator 4 is explained subsequently with reference to FIG. 10 and 11.

FIG. 10 shows the position of the transseptal dilator 4 sticking with its tapered tip in the hole 17 created by laser application in the septal wall 6. In contrast to FIG. 8, the dilator 4 and inner sheath 3 are not immediately advanced into the left atrial cavity 8 because of suspected puncture of a wrong structure. In fact, a blood sample can be taken, pressure measurement can be performed and contrast medium can be injected in order to determine position of penetration.

Eventually, a guide wire 12 may be introduced into the dilator 4 and advanced through the dilator 4 into the left atrial or ventricular cavity 8, 13, as shown in FIG. 11. This allows for conclusive confirmation of a correct position and orientation of the puncture hole 17. In case of correct position of the puncture hole 17, the dilator 4, inner sheath 3 and outer sheath 2 can be advanced over the guide wire 12 through the septum 6 and positioned in the left atrium 8, as shown in FIG. 8 and 9. Subsequently, the guide wire 12 and inner sheath 3 are removed.

As stated before, a simplified transseptal laser puncture can be performed by using only a pre-shaped dilator 4 for guidance of the optical fiber 5 (without using a pre-shaped inner sheath 3 and outer sheath 2). Application of an inner sheath 3 and an outer sheath 2 is not a prerequisite for the TLP procedure, but profoundly eases manipulation of the dilator 4. The single sheath (dilator) technique is preferable particularly in pediatric patients or whenever a less traumatic operation is preferred.

Further, transseptal puncture can be performed merely mechanically by pushing and urging the fiber through the septum without application of laser light. This puncture technique is feasible only in case of a patent fossa ovalis or a very thin membrane of the fossa ovalis is present which is easy to penetrate.

Literature References
1. O'Keefe J H, Vlietstra R E, Hanley P C, et al: Revival of the transseptal approach for catheterization of the left atrium and ventricle. Mayo Clin Proc 1985;60:790-795.
2. Fisher D, Fisher E, Budd J, et al: The incidence of patent foramen ovale in 1,000 consecutive patients. A contrast transesophageal echocardiography study. Chest 1995;107:1504-1509.
3. The transseptal approach for ablation of cardiac arrhythmias: experience of 104 patients. Linker N J, Fitzpatrick A P. Heart 1998;79:379-382.
4. Deshpande S S, Bremner S, Sra J S, et al: Ablation of left free-wall accessory pathways using radiofrequency energy at the atrial insertion site: Transseptal versus transaortic approach J Am Cardiol 1994;5:219-231.
5. Tucker K J, Curtis A B, Murphy J, et al: Transesophageal echocardiographic guidance of transseptal left heart catheterization during radiofrequency ablation of left-sided accessory pathways in humans. Pacing Clin Electrophysiol 1996;19:272-281.
6. Hahn K, Gal R, Sarnoski J, et al: Transesophageal echocardiographically guided atrial transseptal catheterization in patients with normal-sized atria: Incidence of complication. Clin Cardiol 1995;18:217-220.
7. Lesh M D, van Haare G F, Scheinman M M, et al: Comparison of the retrograde and transseptal methods for ablation of left free wall accessory pathways. J Am Coll Cardiol 1993;22:542-549.
8. Baim D S: Percutaneous approach, including transseptal and apical puncture. In Baim D S, Grossman W, eds: Cardiac Catheterization, Angiography, and Intervention, Fifth Edition. William & Wilkins, Baltimore, 1996, pp. 57-81.
9. Daoud E G, Kalbfleisch S J, Hummel J D. Intracardiac echocardiography to guide transseptal left heart catheterization for radiofrequency catheter ablation. J Cardiovasc Electrophysiol 1999;10:358-363.
10. Bazaz R, Schwartzman D. Site-selective atrial puncture. J Cardiovasc Electrophysiol. 2003;14:196-7.

List of Reference Numerals of the Drawings
1 Catheter assembly
2 Outer sheath
3 Inner sheath
4 Dilator
5 Optical fiber
6 Inter-atrial septum
7 Right atrium
8 Left atrium
9 Fossa ovalis
10 Superior vena cava
11 Inferior vena cava
12 Guide wire
13 Left ventricle
14 Right ventricle
15 Distal portion
16 Light emitting surface
17 Transseptal hole
18 Lumen
19 Laser energy source
20 Heart
LA Left atrium
RA Right atrium
LV Left ventricle
RV Right ventricle
IAS Inter-atrial septum
SVC Superior vena cava
IVC Inferior vena cava

What is claimed is:

1. A method for puncture of the septum of a heart, particularly of a human heart, having a septum and several cavities, by means of a laser catheter assembly comprising an optical fiber connectable to a laser energy source, wherein the optical fiber is introduced into a body and advanced to a cavity of the heart and wherein laser light is applied to said optical fiber and aimed at the tissue located in front of the distal end of the fiber so as to create a small hole in the septum.

2. The method of claim 1, wherein the catheter assembly is inserted pervenously into the body, is advanced into the aright atrium and is brought in intimate contact with the interatrial septum.

3. The method of claim 1, wherein the optical fiber is advanced during laser application.

4. The method of claim 1, wherein the laser catheter assembly comprises at least one sheath and the optical fiber, and wherein the optical fiber is advanced beyond a distal end of the sheath before laser application.

5. The method of claim 1, wherein a laser power of 20 W or less preferably about 10 W is applied to the tissue.

6. The method of claim 1, wherein the laser catheter assembly comprises at least one sheath for guiding the optical fiber to the region to be treated.

7. The method of claim 6, wherein the sheath is pre-shaped at a distal end thereof.

8. The method of claim 6, wherein, after puncture of the septal wall, the optical fiber is withdrawn from the at least one sheath and a guide wire is inserted into the sheath and advanced into the left atrium, whereupon the sheath is advanced over the guide wire into the left atrium.

9. A catheter assembly for puncture of the septum of a heart, particularly in the human heart, having a septum and several cavities, comprising at least one longitudinal sheath having an inner lumen extending throughout the sheath and an optical fiber connectable to a laser energy source which is located inside the lumen.

10. The catheter assembly of claim 9, wherein the assembly comprises a dilator housing the optical fiber, an inner sheath overriding the dilator and an outer sheath overriding the inner sheath.
